(19) United States
(12) Patent Application Publication
(10) Pub. No.: US 2007/0269477 A1
(43) Pub. Date: Nov. 22, 2007

(54) Heart Treatment Method

(76) Inventors: Stephen R. Igo, League City, TX (US); James W. Meador, Houston, TX (US)

Correspondence Address: Tim L. Burgess, 402 Oak Lane, Houston, TX, 77024, US

(21) Appl. No.: 11/579,247

(22) PCT Filed: May 5, 2005
(86) PCT No.: PCT/US05/15861

§ 371 Date: Nov. 1, 2006

Related U.S. Application Data
(60) Provisional application No. 60/568,400, filed on May 5, 2004.

(51) Int. Cl. A61K 31/56; A61F 2/06; A61K 31/197; A61K 9/00; A61P 9/14; A61K 36/87; A61K 31/352

(52) U.S. Cl. 424/422; 424/766; 514/169; 514/456; 514/562

(57) ABSTRACT

A method of treating heart disease. Antioxidant agents, such as estradiol, N-acetylcysteine and procyanidins, are administered intra-pericardially to inhibit coronary and myocardial inflammation. The antioxidant agents can be carried in a biodegradable material.

### CROSS REFERENCE TO RELATED APPLICATIONS

This application claims benefit of priority to U.S. Provisional Application Ser. No. 60/568,400, filed May 5, 2004.

### FIELD OF THE INVENTION

The present application is related to compositions and methods for inhibiting inflammation of the coronary blood vessels and heart muscle.

### BACKGROUND OF THE INVENTION

Heart disease is the leading cause of death and disability in industrialized countries of the world. The human and economic toll of heart disease is enormous. According to statistics from the American Heart Association, 12.9 million Americans have a history of coronary heart disease with 7.6 million having suffered a myocardial infarction (heart attack). This year, an estimated 650,000 Americans will have a new coronary attack and about 450,000 will have a recurrent attack. About 47 percent of the people who experience a coronary attack in a given year will die from it. It is estimated that 4.9 million Americans have a history of congestive heart failure with 500,000 new cases diagnosed yearly. The total direct and indirect costs for treating coronary heart diseases are approximately $130 billion yearly.

Coronary artery disease (CAD) is not a single lesion or a single vessel disease; it's pan-coronary. CAD consists of both stenotic fibrotic lesions that are amenable to percutaneous coronary interventions (PCI) (e.g., balloon angioplasty and/or stenting procedures), and non-stenotic highly inflamed plaques that are prone to sudden rupture that is the cause of most heart attacks.

For 2003, it is estimated that nearly one million PCI procedures will be performed in the United States. Initially, balloon angioplasty was utilized to dilate stenotic coronary fibrotic lesions. It was observed that about 40% of the balloon dilated vessels develop reclosure (restenosis) within one year. The restenotic lesion consists of two primary components; intimal hyperplasia due to smooth muscle cell (SMC) proliferation, and constrictive vessel remodeling due to proliferation and/or recruitment of myofibroblasts containing alpha SMC actin with collagen formation in the adventitia surrounding the balloon injury site. The latter restenosis component, vessel constriction, results in greater lumen loss than intimal hyperplasia (Gary et al., Circulation 94:36, 1996; Andersen et al., Circulation 93:1716, 1996). Metallic stent devices can effectively prop open the vessel and prevent constrictive vessel remodeling but are associated with increased intimal hyperplasia resulting in restenosis rates of approximately 25%. Recently developed drug-eluting stents effectively inhibit SMC proliferation and reduce in-stent restenosis rates to less than 10%. The need for employing expensive drug-eluting stent devices might be reduced if constrictive vessel remodeling could be pharmacologically prevented.

Experimental studies have demonstrated that inflammation is a major event associated with balloon angioplasty restenosis, resulting in the recruitment of neutrophils and monocyte/macrophages into the adventitia surrounding the injury site. The inflammatory cells release cytokines (MCP-1 and VCAM-1) and increase reactive oxygen species (ROS) production that stimulates the proliferation and recruitment of myofibroblasts leading to constrictive vessel remodeling (Wilcox et al., Ann N Y Acad Sci 947:68, 2001; Okamoto et al., Circulation 104:2228, 2001; Mori et al., Circulation 105:2905, 2002). Clinical studies have also demonstrated that inflammation plays a pathogenic role in the development of restenosis after coronary balloon angioplasty. Patients with vessel restenosis, compared with nonrestenotic patients, had elevated plasma levels of MCP-1 that correlated with increased monocyte/macrophage activity and ROS production. Multivariate regression analysis showed that the plasma MCP-1 level, measured 15 days after balloon angioplasty, was the only independent predictor of vessel restenosis at six month follow-up (Cipollone et al., Arterioscler Thromb Vasc Biol 21:327, 2001).

Recently, there has been a paradigm shift in cardiovascular medicine as to the cause of acute coronary syndromes (unstable angina, myocardial infarction). Previously it was believed that a myocardial infarction resulted from the gradual build-up of plaque within the coronary artery wall that eventually blocked the flow of blood to the heart muscle causing a heart attack. Recent studies have shown that most heart attacks occur not because of progressive vessel stenosis, but because of acute rupture of a non-stenotic atherosclerotic plaque (also termed vulnerable plaque). The structural characteristics of vulnerable non-stenotic atherosclerotic plaque include eccentric outward vessel remodeling with a large lipid pool covered by a thin cap of fibrous tissue. Lipids in the coronary artery wall attract monocytes and as macrophage-derived foam cells build up a cascade of inflammation occurs with local expression of cytokines and activation of enzymes, such as matrix-degrading proteinases, that eventually leads to the breakdown of the cells in the thin fibrous cap over the lipid pool. When the cap erodes far enough, a rupture occurs that causes an occlusive thrombus in the bloodstream to develop leading to a heart attack (Shah, J Am Coll Cardiol 41:S15, 2003; Shah, Prog Cardiovasc Dis 44:357, 2002; Libby, Am J Cardiol 88:3J, 2001; Shah, Cardiol Rev 8:31, 2000; Gronholdt et al., Eur Heart J 19:C24, 1998; Shah, Vasc Med 3:199, 1998). Passivation of vulnerable plaque represents a therapeutic concept by which the structure or content of the atherosclerotic plaque is changed to reduce the risk of subsequent rupture and thrombosis using strategies that address different components of the plaque or the endothelium. Reducing macrophage infiltration, accumulation of inflammatory cells, secretion of enzymes that cause degradation of the fibrous cap, and lipid content may reduce the risk of atherosclerotic plaque rupture (Monroe et al: J Am Coll Cardiol 41:S23, 2003; Shah et al., Cardiol Clin 14:17, 1996).

Recent clinical studies have demonstrated that for patients with myocardial infarction, all three major coronary arteries are widely diseased with multiple vulnerable plaque sites. In addition, acute coronary syndrome is associated with inflammation and neutrophil activation throughout the coronary vascular bed resulting in multiple unstable lesions (Rioufol et al., Circulation 106: 804, 2002; Buffon et al., N Engl J Med 347:5, 2002; Asakura et al., J Am Coll Cardiol 37:1284, 2001; Goldstein et al., N Engl J Med 343:915, 2000).

Oxidative stress and the production of intracellular oxygen free radicals or reactive oxygen species (ROS) have been implicated in the pathogenesis of a variety of diseases (Kunsch et al., Circ Res 85:753, 1999; Laroia et al., Int J Cardiol 88:1, 2003). In excess, ROS and their byproducts can overpower endogenous antioxidant defense mechanisms and cause oxidative damage to biological macromolecules, such as DNA, protein, carbohydrates, and lipids and can be cytotoxic. An increasing body of evidence suggests that oxidant stress is involved in the pathogenesis of many cardiovascular diseases including atherosclerosis, constrictive vessel remodeling following balloon angioplasty, ischemia-reperfusion injury after myocardial infarction, and congestive heart failure. A variety of cardiovascular cell types including neutrophils, macrophages, fibroblasts, smooth muscle cells (SMCs), and endothelial cells are known to produce and release ROS. The endothelium maintains vascular homeostasis by the production and release of nitric oxide. Vascular diseases are characterized by impaired endothelium-derived NO bioactivity that may contribute to clinical cardiovascular events. Growing evidence indicates that impaired endothelium-derived NO bioactivity is due, in part, to excess vascular oxidative stress (Thomas et al., Antioxid Redox Signal 5:181, 2003).

Atherosclerosis is a chronic inflammatory disease characterized by the accumulation of mono-nuclear leukocytes, SMCs, lipids, and extracellular matrix components in the arterial wall (Libby et al., Am J Cardiol 91:3A, 2003; Ross, Annu Rev Physiol 57:791, 1995). One of the earliest detectable events in the generation of atherosclerosis is the focal infiltration of inflammatory cells into the arterial wall and their transformation into lipid-laden macrophages (foam cells). A variety of proinflammatory or pro-oxidant factors stimulate vascular cells to generate ROS. These ROS serve as second-messenger molecules that signal the expression of atherogenic gene products (Egashira, Hypertension 41:834,2003; Werle, et al., Cardiovasc Res 56:284, 2002; Viedt et al., Arterioscler Thromb Vasc Biol 22:914, 2002; Ikeda et al., Clin Cardiol 25:143, 2002; Shin et al., Atherosclerosis 160:91, 2002; Marui et al., J Clin Invest 92:1866, 1993) such as vascular cell adhesion molecule-1 (VCAM-1) and monocyte chemoattractant protein-1 (MCP-1) through a redox-sensitive mechanism involving the redox-regulated transcription factor nuclear factor-kappaB (NF-kB). ROS-induced expression of these inflammatory gene products promotes the infiltration of monocytes into the vessel wall with the local release of additional pro-inflammatory signals and exacerbation of endothelial cell dysfunction. Conversely, chemical or cellular antioxidants protect vascular cells against oxidative stress by scavenging ROS or by modulating the redox-sensitive signaling pathways and blocking atherogenic gene expression.

Considerable evidence suggests that ROS are also involved in the pathogenesis of cardiovascular diseases such as myocardial ischemia-reperfusion injury (Chen et al., J Biol Chem 278:36027, 2003; Lubbers et al., J Cardiovasc Pharmacol 41:714, 2003; Li et al., J Am Coll Cardiol 41:1048, 2003; Becker et al., Z Kardiol 89:Suppl 9, 88, 2000; Bassenge et al., Am J Physiol 279:H2431, 2000; McDonald et al., Free Radic Biol Med 27:493, 1999; Vanden Hoek et al., J Mol Cell Cardiol 29:2571, 1997) and congestive heart failure (Byrne et al., Arch Mal Coeur Vaiss 96:214, 2003; Heymes et al., J Am Coll Cardiol 41:2164, 2003; Maack et al., Circulation 108:1567, 2003; Hunt et al., Am J Physiol 283:L239-45, 2002; Choudhary and Dudley, Congest Heart Fail 8:148, 2002; Sorescu and Griendling, Congest Heart Fail 8:132, 2002; Lopez-Farre and Casado, Hypertension 38:1400, 2001; Dhalla et al., J Hypertens 18:655, 2000; Ide et al., Circ Res 86:152, 2000; Cai and Harrison, Circ Res 87:840, 2000; Ferrari et al., Eur Heart J 19:B2, 1998).

ROS can diminish myocardial contractile function and cause lipid peroxidation of membrane phospholipids, which ultimately leads to myocyte structural damage. Recently, ROS have been suggested to be involved in apoptosis (cell death), which might play an important role in the pathogenesis of heart failure. Moreover, ROS can cause endothelial dysfunction and induce arrhythmia, both of which may contribute to the progression of heart failure. Therefore, oxidant stress plays an important role in myocardial failure (Tomomi et al., Circ Res 86:152, 2000).

### DETAILED DESCRIPTION OF THE INVENTION

The present invention provides a method for treating heart disease which comprises administering intrapericardially at a therapeutically effective dosage a non-endothelium-derived antioxidant agent capable of scavaging reactive oxygen species or inhibiting superoxide production or both. The intrapericardial administration may by injection or infusion of the antioxidant agent(s). The method is effective to treat diseases and/or injury of the heart or coronary vasculature, for example, high risk atherosclerotic plaque, angioplasty constrictive vessel remodeling, myocardial ischemia-reperfusion injury, or congestive heart failure.

“Antioxidant agents” includes any steroid hormone, amino acid, protein, chemical, or other molecule that increases nitric oxide bioavailability within cardiovascular cells by scavaging reactive oxygen species stimulating nitric oxide production or bioactivity, decreasing superoxide production, or both. The antioxidant agent(s) is delivered intrapericardially, either with or without a biodegradable or non-biodegradable carrier (e.g., non-polymeric or polymeric material) in order to treat or prevent disease.

In one embodiment of the invention, the therapeutic antioxidant agent is estradiol, a natural non-endothelium-derived steroid hormone that promotes nitric oxide and prostacyclin production in vascular cells and inhibits cytokine-induced superoxide expression.

Estradiol is a naturally occurring, nontoxic, small molecule (mw 272.4), hydrophobic, lipophilic, 18-carbon steroid hormone. Estradiol is the most potent form of estrogen and is a generic drug. The anti-atherogenic and cardioprotective effects of estrogen are well recognized (White, Vascul Pharmacol 38:73, 2002; Mendelsohn, Am J Cardiol 90:3F, 2002; Mendelsohn, N Engl J Med 340:1801, 1999; Farhat et al., FASEB J 10:615, 1996). Studies have shown that estradiol may play an important role in preventing or reversing endothelial dysfunction associated with atherosclerosis (Rubanyi et al., Vascul Pharmacol 38:89, 2002; Rodriguez et al., Life Sci 71: 2181, 2002). Stimulation of endothelial cells with estradiol causes a rapid and dose-dependent release of nitric oxide and prostacyclin (Sherman et al., Am J Respir Cell Mol Biol 26:610, 2002; Alvarez et al., Circ Res 91:1142, 2002; Sumi et al., Life Sci 69:1651, 2001; Nuedling et al., Cardiovasc Res 43:666, 1999), and inhibits endothelin-1 synthesis (Dubey et al., Hypertension 37:640, 2001). Stimulation of endothelial Fas ligand expression by estradiol inhibits the migration of inflammatory cells into the vessel wall (Amant et al., Circulation 104:2576, 2001). Estradiol has been shown to inhibit the expression of vascular inflammatory cytokines in atherosclerotic plaque. Monocyte/macrophage infiltration to the arterial wall is an initial step in atherosclerosis, and MCP-1 is thought to play a central role in the recruitment of these cells. Estradiol suppresses vascular MCP-1 expression in vivo (Ryomoto et al., J Vasc Surg 36:613, 2002) and decreases macrophage recruitment in atherosclerotic plaque (Ryomoto et al., J Vasc Surg 36:613, 2002; Seli et al., Menopause 8:296, 2001; Pervin et al., Arterioscler Thromb Vasc Biol 18:1575, 1998).

Estradiol protects against atherosclerosis independent of changes in plasma lipoproteins. Estradiol modulates the vascular inflammatory response by inhibiting cytokine production, the cytokine-induced expression of cell adhesion molecules (Caulin-Glaser et al., J Clin Invest 98:36, 1996), and platelet aggregation and adhesion. (Joswig et al., Exp Clin Endocrinol Diabetes 107:477, 1999; Nakano et al., Arterioscler Thromb Vasc Biol 18:961, 1998). Estradiol reduced atherosclerotic plaque size and increased endothelial nitric oxide production in hyper-cholesterolemic rabbits with severe endothelial cell dysfunction (Nascimento et al., Am J Physiol 276: H1788, 1999).

Administration of estradiol protects against endothelial and myocardial dysfunction following ischemia/reperfusion injury. Estradiol acts as an antioxidant by improving the nitric oxide/superoxide balance in the vessel wall, increasing nitric oxide bioavailability (Wagner et al., FASEB J 15:2121, 2001) and normalizing the expression of anti-inflammatory factors in endothelial cells. Beneficial cardiovascular effects of estradiol include vasodilation (Thompson et al., Circulation 102:445, 2000; Lamping et al., Am J Physiol 271:H1117, 1996; Keaney et al., Circulation 89:2251, 1994), inhibition of response to vascular injury (Delyani et al., J Mol Cell Cardiol 28:1001, 1996), limiting myocardial infarct size (Lee et al., J Mol Cell Cardiol 32: 1147, 2000; Smith et al., Circulation 102:2983, 2000), and reducing reperfusion arrhythmias (Tsai et al., J Pharmacol Exp Ther 301:234, 2002; Kim et al., Circulation 94:2901, 1996).

Estradiol has dual beneficial effects for treating coronary angioplasty restenosis by inhibiting SMC proliferation and promoting healing of the endothelial cell lining of the artery (Yue et al., Circulation 102:III281, 2000), and reduction in MCP-1 expression and macrophage accumulation (Ryomoto et al., J Vasc Surg 36:613, 2002). The effects are mediated by the expression of vasoprotective genes and by an increase in the production of endothelial-derived factors nitric oxide and prostacyclin. Experimental studies using the porcine coronary model have demonstrated that estradiol-eluting stents reduce neointimal formation (SMC proliferation) by nearly 40% with no delay in vascular repair (endothelial cell regrowth), even with time-limited (<24 hours) drug release (New et al., Catheter Cardiovasc Interv 57:266, 2002). Intramural estradiol delivery via perfusion balloon catheter (also time-limited) inhibits SMC proliferation (Chandrasekar et al., J Am Coll Cardiol 36:1972, 2000) and promotes endothelial cell regrowth by effecting mitogen-activated protein kinase (MAPK) activity (Geraldes et al., Arterioscler Thromb Vasc Biol 22:1585, 2002) and endothelial cell nitric-oxide synthase expression (Chandrasekar et al., J Am Coll Cardiol 38:1570, 2001) in porcine model of balloon angioplasty restenosis.

Methods for coating or chemically bonding 17 beta-estradiol to the surface of various implants including stents, artificial cardiac valves and catheters to inhibit local SMC growth and stimulate endothelial cell growth (reduce restenosis) and to improve the antithrombogenicity and biocompatibility of such implants has been described (U.S. Pat. Nos. 6,617,027 and 6,383,215).

U.S. Pat. Nos. 6,350,739 and 6,172,056 describe pharmaceutical compositions and methods for the prevention and treatment of ROS-mediated ischemic cell damage. The '739 Patent includes the method of systemically administering an estrogen compound for treating stroke and other ischemic syndromes. The '056 Patent includes the method of systemically administering a steroid drug (e.g., estradiol) and at least one pharmaceutical adjuvant to inhibit changes in cells and tissues, such as lipid peroxidation and low-density lipoprotein oxidation, and reduce cell membrane and endothelial damage.

In another embodiment of the invention, the therapeutic antioxidant agent is N-acetylcysteine, a natural non-endothelium-derived amino acid that increases vascular nitric oxide bioavailability by scavenging reactive oxygen species and inhibiting cytokine-induced superoxide expression.

N-acetylcysteine (NAC) is a naturally occurring, nontoxic, small molecule (mw 163.2), thiol-containing amino acid. NAC is a potent antioxidant (ROS scavenger) and glutathione enhancer that increases nitric oxide bioavailability. NAC is a generic drug that has been in clinical use for more than 30 years. NAC has been shown to inhibit the expression of vascular inflammatory cytokines in atherosclerotic plaque. Experimental studies have shown that NAC decreases the matrix-degrading capacity of macrophage-derived foam cells in atherosclerotic lesions (Galis et al., Circulation 97:2445, 1998). NAC decreased gelatinolytic activity and gelatinase (metalloproteinase) expression by foam cells. In vulnerable atherosclerotic plaque, activated T-lymphocytes and platelets release high amounts of CD40 ligand (CD40L) contributing to plaque instability and thrombus formation that leads to acute coronary syndromes. CD40L inhibits endothelial cell regrowth and reduces nitric oxide bioavailability by stimulating ROS production. NAC has been shown to reverse these effects (Urbich et al., Circulation 106:981, 2002). NAC inhibits cytokine-induced MCP-1 expression in endothelial cells (Lee et al., Am J Physiol 284:H185, 2003) and NF-kB production in SMCs (Hayashi et al., Neurol Res 23:731, 2001; Ishizuka et al., Clin Exp Immunol 120:71, 2000). NAC attenuates cytokine-induced p38 MAP kinase activation in endothelial cells (Hashimoto et al., Br J Pharmacol 132:270, 2001) and VCAM-1 and E-selection adhesion molecule expression (Faruqi et al., Am J Physiol 273:H817, 1997). NAC enhances the coronary vasodilation and antiplatelet effects of nitric oxide donor drug (Chirkov et al., J Cardiovasc Pharmacol 28:375, 1997; Pizzulli et al., Am J Cardiol 79:28, 1996; Stamler et al., Circ Res 65:789, 1989). Taken together, the antioxidant effects of NAC willy help stabilize vulnerable atherosclerotic plaque.

Reperfusion of ischemic myocardium is associated with rapid and sustained release of oxygen-derived free radicals leading to peroxidation of lipids and depletion of endogenous antioxidants. These factors contribute to the development of reperfusion injury, characterized by temporary impairment of systolic function (myocardial stunning), arrhythmias, and possibly further necrosis. Administration of NAC protects against endothelial and myocardial cell dysfunction following ischemia/reperfusion injury (Dhalla et al., Cardiovasc Res 47:446, 2000; Ferrari et al., Am J Med 91:95S, 1991; Sochman et al., Int J Cardiol 28:191, 1990; Ceconi et al., J Mol Cell Cardiol 20:5, 1988). In patients with evolving myocardial infarction, NAC in combination with nitroglycerin and streptokinase was associated with significantly less oxidative stress, a trend toward more rapid reperfusion, and better preservation of left ventricular function (Sochman et al., Clin. Cardiol 19: 94, 1996; Arstall et al., Circulation 92:2855, 1995).

In another embodiment of the invention, the therapeutic antioxidant agent is procyanidin with or without bonded gallic acid.

Procyanidin is a naturally occurring, organic compound found in approximately 80% of woody plants and 20% of leguminous plants. Also known as proanthocyanidin, these compounds are part of a specific group of polyphenolic compounds called flavonoids. Flavonoids are further categorized by subgroups. Procyanidins belong to the category known as condensed tannins. Esterification of flavanols (−)-epicatechin and procyanidin B2 by gallic acid increases the free radical scavenging ability of these compounds. The dimeric proanthocyanidins having the C₄-C₈ linkage have greater free radical scavenging activity that the C₄-C₆ linkage, and these gallate esters are only found in grape seed extract form. Grape seed extract contains oligomeric proanthocyanidin complex's made up of dimers or trimers of (+)-catechin and (−)-epicatechin. The procyanidin dimers are comprised of procyanidins B1, B2, B3, B4, B5, B6, B7 and B8. There are six procyanidin trimers which include procyanidin C1 and C2. Additionally, several gallolyl procyanidins (which are most commonly the gallate esters of thee dimeric procyanidins and some free gallic acid) are present (Bombardelli et al., Fitoterapia 1995;66:291-317 and da Silva et al., Phytochemistry 1991;30:1259-1264).

Procyanidins are chemical compounds in which catechins and/or epicatechins are linked. There may or may not be attached gallate ester groups. The biological properties of flavonoids, including procyanidins (also known as proanthocyanidins) have been extensively reviewed (Bagchi et al., Res Commum Mol Pathol Pharmacol 1997; 95:179-189, Havsteen et al., Biochem Pharmacol 1983;32:1141-1148, Frankel et al., Lancet 1993; 341:454-457). Like all other polyphenols, procyanidins display strong antioxidant activity. In vitro, procyanidins are powerful inhibitors of tyrosine nitration by peroxinitrate. Procyanidins have been shown to have cardio-protective effects (Aldini et al. Life Sci. Oct. 17, 2003;73(22):2883-98 and Bombardelli et al. Fitoterapia 1995; 66(4):291-317). Oxidative modification was also shown to play a key role in the initiation of atherogenesis and flavonoids prevent LDL oxidation in vitro by scavenging free radicals (Miller et at, Arch. Bio. Biophys, 1995,322,339-46).

The preferred chemicals are procyanidins with attached gallic esters. However, monomers and/or oligomers of catechin and epicatechin without esterified gallic acid or mixtures thereof can be used.

The procyanidin gallates have increased antioxidant potential which may be due to the additional three hydroxyl groups contributed by the gallic acid, but may also reflect structural properties of the ester bond in these compounds.

As noted above, the present invention provides methods and compositions for region specific administration of antioxidant agents for treating or preventing diseases and/or injury of the heart or coronary vasculature (e.g., vulnerable atherosclerotic plaque, ischemic-reperfusion injury, constrictive vessel remodeling, congestive heart failure, intimal hyperplasia or a combination thereof) comprising the step of intrapericardial injection or infusion of an antioxidant agent(s).

The pericardial sac is a thin fibrous membrane that encloses the heart, effectively creating a natural reservoir for drug delivery. Coronary arteries located on the surface of the heart are constantly bathed in pericardial fluid. U.S. Pat. Nos. 5,681,278 and 5,900,433 describe a method for treating blood vessels in humans, comprising the steps of: (a) selecting a congener of an endothelium-derived bioactive agent (e.g., nitric oxide or prostacyclin); (b) administering a therapeutically effective dosage of the selected congener to a site proximately adjacent to the exterior of a coronary blood vessel (e.g., intrapericardial, IPC); and (c) allowing the congener to treat the coronary blood vessel from the outside-in. In vivo studies demonstrated that the IPC delivery of a nitric oxide donor drug prevented vessel thrombosis and occlusion in a canine model of coronary artery injury and stenosis (Willerson et al., Tex Heart Inst J 23:1, 1996). In addition, the IPC drug delivery method was shown to be safer and more effective than intravenous (e.g., systemic) drug infusion in a dose response manner. An advantage of the IPC method is that a drug combined with a controlled release material can provide prolonged drug delivery (e.g. days to weeks) to the coronary arteries following a single IPC injection.

Other investigators have validated the IPC delivery method for administration of antiarrhythmic (Ayers et al., J Cardiovasc Electrophysiol 7:713, 1996; Fei et al., Circulation 96: 4044, 1997; Moreno et al., J Cardiovasc Pharmacol 36:722, 2000; Kumar et al., J Am Coll Cardiol 41:1831, 2003), angiogenic (Laham et al., J Thorac Cardiovasc Surg 116:1022, 1998; Laham et al., Curr Interv Cardiol Rep 2:213, 2000; Laham et al., J Pharmacol Exp Ther 292: 795, 2000; Laham et al., Catheter Cardiovasc Interv 58:375, 2003), antirestenosis (Makkar et al., Circulation 98:I-399 [abstract 2098], 1998; Kaul et al., J Am Coll Cardiol 33:88A [abstract 1190-134], 1999; Kaul et al., J Am Coll Cardiol 33:71A [abstract 845-6], 1999; Hou et al., Circulation 102:1575, 2000; Baek et al., Circulation 105:2779, 2002), vasodilator (Waxman et al., J Am Coll Cardiol 33:2073, 1999), and gene therapy agents (Lamping et al., Am J Physiol 272:H310, 1997; Fromes et al., Gene Ther 6:683, 1999; Zhang et al., J Mol Cell Cardiol 31:721, 1999; March et al., Clin Cardiol 22:123, 1999).

U.S. Pat. No. 6,333,347 describes a method for the IPC delivery of time release micro-tubule agents (e.g. anticancer drug paclitaxel) for treating the pericardium, heart, and coronary vasculature. This class of drugs is known to inhibit cell proliferation.

Pharmacokinetic studies have demonstrated that IPC drug administration increases drug concentration in the coronary wall, prolongs the drug redistribution time, and reduces systemic drug effects, compared to local intramural and intravenous drug delivery (Stoll et al., Clint Cardiol 22:I10, 1999; Hermans et al., J Pharmacol Exp Ther 301:672, 2002; Ujhelyi et al., J Cardiovasc Electrophysiol 13:605, 2002; Gleason et al., J Cardiovasc Magn Reson 4:311, 2002).

Preferably, methods and compositions of the present invention are fashioned in a manner appropriate to the intended use. Within certain aspects of the present invention, the therapeutic composition should be biocompatible, and release one or more therapeutic agents over a prescribed time period. For example, fast release therapeutic compositions provide an initial burst release of 10% to 25% of a therapeutic agent (e.g., estradiol and/or N-acetylcysteine and/or procyanidin) for a period of up to 2 days with continuous release thereafter for a period of up to 45 days. Within other embodiments, slow release therapeutic compositions provide continuous release of a therapeutic antioxidant agent(s) over a period of up to 45 days. Furthermore, the therapeutic compositions of the present invention should preferably be stable for several months and be capable of being produced and maintained under sterile conditions.

The antioxidant agent can be administered intrapericardial in a dosage to achieve a therapeutic result. In one embodiment, an antioxidant agent such as estradiol is administered at a dosage ranging from 10 to 100 ug/kg (body weight)/day for a treatment duration ranging from 1 to 45 days. In another embodiment, an antioxidant agent such as N-acetylcysteine is administered at a dosage ranging from 10 to 100 mg/kg (body weight)/day for a treatment duration ranging from 1 to 45 days. In another embodiment, an antioxidant agent such as procyanidin is administered at a dosage ranging from 10 to 100 ug/kg (body weight)/day for a treatment duration ranging from 1 to 45 days. With any of these embodiments, the antioxidant agent (e.g., estradiol and/or N-acetylcysteine and/or procyanidin) may be administered along with other therapeutic agents (e.g., statins).

Within one preferred embodiment of the invention, the therapeutic antioxidant agent (estradiol and/or N-acetylcysteine and/or procyanidin) is delivered intrapericardial via a controlled release carrier (SABER™, Durect Corporation, Cupertino, Calif.). The SABER™ delivery system is a non-polymeric gel material that can be formulated with small and large molecule drugs. The SABER™ system can provide continuous drug release for up to three months following a single injection. A major advantage of SABER™, compared to microsphere and polymer-based delivery systems, is that the drug and delivery gel does not have to be manufactured together. The antioxidant drug and SABER™ delivery gel can be produced and packaged separately and are mixed together by the physician just prior to intrapericardial injection. Preclinical animal studies, conducted by Durect Corporation, have shown that the SABER™ system is biocompatible when injected into the pericardial space. Estradiol and N-acetylcysteine, being small molecule lipophilic drugs, are ideally suited for incorporation with the SABER™ time release gel and for transcoronary drug diffusion and retention following pericardial administration.

Within certain embodiments of the invention, the antioxidant agents may be formulated along with other compounds or compositions, such as, for example, a gel, wrap, implant, fiber, microsphere, or the like. Within certain embodiments the compound or composition may function as a carrier, which may be either polymeric, or non-polymeric. Representative examples of polymeric carriers include poly (ethylene vinyl acetate), copolymers of lactic acid and glycolic acid, poly (caprolactone), poly (lactic acid), copolymers of poly (lactic acid) and poly (caprolactone), gelatin, hyaluronic acid, collagen matrices, celluloses and albumen.

Intrapericardial administration of antioxidant agent(s) with or without a controlled release carrier may be accomplished by a variety of methods and devices. Within one preferred embodiment of the invention, the antioxidant agent(s) or composition (e.g., antioxidant drugs and controlled release carrier) may be administered transatrial through the right atrium (see U.S. Pat. Nos. 5,269,326; 5,968,010 and 6,200,303). Briefly, the catheter device is designed for conventional percutaneous insertion via the femoral vein. For this method, a guide catheter is advanced into the right atrium and the atrial wall is pierced with a micro-catheter that allows pericardial fluid withdrawal and/or drug injection. The device has undergone extensive preclinical testing and been shown to be a safe and effective device for catheterizing the normal pericardial space (Verrier et al., Circulation 98:233, 1998; Waxman et al., Catheter Cardiovasc Interv 49:472, 2000; Pulerwitz et al., J Interv Cardiol 14:493, 2001). In another embodiment, a transthoracic catheter device (PerDUCER™, Comedicus Inc., Columbia Heights, Minn.) can be used for pericardial access (see U.S. Pat. Nos. 5,827,216 and 6,162,195). Briefly, the PerDUCER™ device is designed for percutaneous, substernal, insertion, and uses a novel suction tip and sheathed needle that provides pericardial capture and puncture, respectively, without injury to the heart. The PerDUCER™ device has undergone extensive testing in animals, initial clinical evaluations, and is approved for sale in Europe (Macris and Igo, Clin Cardiol 22:I36, 1999; Tio et al., Int J Cardiol 82:117, 2002; Hou and March, J Invasive Cardiol 15:13, 2003). Catheter devices for transventricular access of the pericardial space via the right ventricle (see U.S. Pat. No. 5,797,870) and left ventricle (see U.S. Pat. No. 6,238,406) for the injection of growth factors or gene products for angiogenesis (blood vessel growth) has been described.

We claim:

1. A method for treating heart disease, which comprises administering intrapericardially at a therapeutically effective dosage a non-endothelium-derived antioxidant agent capable of scavaging reactive oxygen species or inhibiting superoxide production or both.

2. The method of claim 1 in which said antioxidant agent is estradiol, N-acetylcysteine, a procyanidin, or a combination thereof.

3. The method of claim 2 in which said procyanidin has attached gallic esters.

4. The method of claim 2 in which said procyanidin is an oligomer of monomeric flavanols

5. The method of claim 4 in which said monomeric flavanols are (+)-catechin and (−)-epicatechin.

6. The method of claim 2 in which said procyanidin is an extract of grape seed.

7. The method of claim 1 in which said step of administering includes delivering said antioxidant agent in a controlled manner over a sustained period of time.

8. The method of claim 1 in which said dosage is a rate from about 10 to about 100 μg/kg (body weight)/day for from 1 to 45 days.

9. The method of claim 1 in which said step of administering comprises intrapericardially infusing said antioxidant agent transatrially through a percutaneously inserted catheter.

10. The method of claim 1 in which said step of administering comprises intrapericardially infusing said antioxidant agent transventrically through a percutaneously inserted catheter.

11. The method of claim 1 in which said step of administering comprises intrapericardially injecting said antioxidant agent transthoracically through a percutaneously inserted needle.

12. The method of claim 1 in which said step of administering comprises inserting an extravascular implant capable of extended time release of said antioxidant agent.

13. The method of claim 12 in which said implant is a biodegradable controlled-release carrier carrying said antioxidant agent.

14. The method of claim 13 in which said carrier is a non-polymeric gel that can be mixed with said antioxidant agent immediately prior to intrapericardial administration.

15. The method of claim 1 in which said dosage is effective to provide the therapeutic effects of preventing or treating vulnerable atherosclerotic plaque, ischemic-reperfusion injury, constrictive vessel remodeling, congestive heart failure, intimal hyperplasia, or a combination thereof.
